(19) United States
(12) Patent Application Publication
(10) Pub. No.: US 2022/0128517 A1
(43) Pub. Date: Apr. 28, 2022

(54) Focal Point Determination Based on Nonlinear Mixing of Sound Beams

(71) Applicant: Alien Sandbox, LLC, Austin, TX (US)

(72) Inventors: Neal Allen Hall, Austin, TX (US); Randall Paul Williams, Austin, TX (US); Ehsan Vatankhah, Austin, TX (US)

(21) Appl. No.: 17/505,195

(22) Filed: Oct. 19, 2021

Related U.S. Application Data
(60) Provisional application No. 63/105,024, filed on Oct. 23, 2020.

(51) Int. Cl. G01N 29/24; G10K 11/24; A61B 8/08; G01S 15/89; A61B 8/00

(52) U.S. Cl. CPC G01N 29/2475; G10K 11/24; A61B 8/5207; A61B 8/4263; A61B 8/4488; A61B 8/4494; G01S 15/8915

(57) ABSTRACT

Systems, methods, and mechanisms for sound beam focal point determination within an acoustic medium may include propagating a first sound beam to intersect a second sound beam, where the second sound beam converges at a focal point. The first and second sound beams may originate from known locations. A direction of the first sound beam within the acoustic medium may be adjusted to produce a maximum amplitude of signals generated from nonlinear mixing of the first sound beam and the second sound beam, where the maximum amplitude may correspond to the intersection of the first sound beam with the focal point of the second sound beam. A location of the intersection may be determined, at least in some instances, based on the known locations, the adjusted direction of the first sound beam, and a direction of the second sound beam.

### PRIORITY DATA

This application claims benefit of priority to U.S. Provisional Application Ser. No. 63/105,024, titled “Focal Point Determination Based on Nonlinear Mixing of Sound Beams”, filed Oct. 23, 2020, which is hereby incorporated by reference in its entirety as though fully and completely set forth herein.

### FIELD OF THE INVENTION

This disclosure relates generally to systems, methods, and mechanisms for sound beam focal point determination within an acoustic medium, e.g., based on nonlinear mixing of sound beams in the acoustic medium.

### DESCRIPTION OF THE RELATED ART

In existing implementations, sound waves may be used in various medical and industrial applications, e.g. for fetal sonograms and other common use-cases of medical imaging. In addition to imaging applications, focused sound beams can be used in medical ultrasound in lithotripsy (the breaking up of kidney stones using high intensity ultrasound) and in histotripsy (the liquefaction of blood clots). Additionally, sound waves may also be used to image the earth's sub-surface on land and/or at sea, e.g., as in the case of geological exploration.

In many areas of acoustics, the need arises to measure properties of a sound field or a focused sound beam within a medium, without inserting sensors into the medium. For example, consider the specific case of a focused ultrasound in a brain. Focused ultrasound beams in the brain are used in histotripsy and are also being explored to control or modulate neurological behavior, e.g., with the hope that ultrasound can be used to treat neurological diseases such as Parkinson's disease and obsessive-compulsive disorder. In such implementations, a typical operating frequency is 0.5 megahertz (MHz), which corresponds to an acoustic wavelength of approximately 3 millimeters (mm). In a typical scenario, a transducer approximately 2 cm in diameter is mounted on the outside surface of a skull to generate an ultrasound beam that focuses down to a region in the brain with approximately 3 mm diameter. In principal, such a beam can be steered and the focal point moved in space to a targeted region of the brain. Ultrasound beam steering is accomplished by inserting a lens into the transducer or, more frequently, by using a phased-array transducer. The nonuniformity in skull thickness and the contrast between the acoustical properties of the skull and underlying soft tissue introduce aberrations into the path of the ultrasonic beam. As a consequence, a major challenge in the field is knowing precisely where the focal point is located within the brain and knowing what sound pressure is achieved at the focal point. Such information may be required to compute dosing (e.g., to answer the questions of what part of brain was treated with ultrasound and at what sound pressure or sound intensity was that part of the brain treated). Models have been introduced that simulate propagation and focusing of ultrasound beams to predict the location of the focal point and the peak pressure achieved, but models are not as reliable as a direct observation or measurement of the sound field. In some in vitro scenarios (e.g. a fake or gel-like brain replica used in a laboratory), one has the ability to insert an acoustic measurement device (e.g. a small needle-like hydrophone) into the medium and directly measure the sound field. In such case, the probe can be moved about to measure sound pressure at any point. However, in many other scenarios (e.g. in vivo scenarios such as a living brain), sticking needle-like hydrophone probes into the brain is not possible.

Likewise, in the case of subsurface imaging of the earth and/or in any solid, it may not be practical to probe the field with instruments simply because a probe cannot be easily inserted into a solid. In other scenarios such as focused beams at sea, one does not have the ability to instrument the entire field of interest (e.g. the entire ocean) with hydrophones.

Therefore, further improvements in the field are desired.

### SUMMARY OF THE INVENTION

Various embodiments of systems, methods, and mechanisms for sound beam focal point determination within an acoustic medium, e.g., based on nonlinear mixing of sound beams in the acoustic medium, are described herein.

For example, in some embodiments, determining properties of an interacting region of intersecting sound beams within an acoustic medium may include propagating a first sound beam through the acoustic medium and adjusting at least one of a direction or focal length of the first sound beam such that the first sound beam intersects a second sound beam propagated through the acoustic medium. The intersection of the first sound beam and the second sound beam may generate nonlinear mixing of the first sound beam and the second sound beam. Additionally, signals resulting from the nonlinear mixing of the first sound beam and the second sound beam may be received from at least one sensor. The signals may be representative of sound pressure in the acoustic medium. Additionally, the signals representative of the sound pressure may be processed to determine properties of the interacting region, e.g., such as a location of an intersections of the first sound beam and the second sound beam. In at least some embodiments, the processing the signals may include performing a time-of-arrival analysis.

As another example, in some embodiments, determining a location of an interacting region of intersecting sound beams within an acoustic medium may include propagating a first sound beam in a first direction through the acoustic medium from a first location and adjusting the first direction to maximize an amplitude of signals generated from nonlinear mixing of the first sound beam and a second sound beam propagated through the acoustic medium in a second direction from a second location. Additionally, upon detection of the maximum amplitude of the signals generated from nonlinear mixing of the first sound beam and a second sound beam, the location of the interacting region may be determined based, at least in part, on the first location and the adjusted first direction. The first location may be within a reference frame and may corresponds to a first source generating the first sound beam.

As an additional example, in some embodiments, a first sound beam and a second sound beam may be propagated (e.g., via the same and/or independent systems) through an acoustic medium and signals representative of sound pressure in the acoustic medium may be received from at least two sensors. The signals may be processed to determine properties of an interacting region of the first sound beam and the second sound beam, including determining a location of an intersection of the first sound beam and the second sound beam in the acoustic medium. In some embodiments, the first sound beam may converge at a first focal point and the second sound beam may converge at a second focal point. Further, a location of the first focal point within the acoustic medium may be adjusted (e.g., swept and/or moved throughout the acoustic medium), e.g., based, at least in part, on the received signals, to produce a maximum amplitude of signals created (e.g., generated) from nonlinear mixing of the first sound beam and the second sound beam. In some embodiments, to adjust the location of the first focal point within the acoustic medium, at least one of a direction of the first sound beam, a frequency of the first sound beam, an amplitude of the first sound beam, a depth of the first focal point, and/or a focal length of the first sound beam may be adjusted. Note that in some embodiments, transducers configured to propagate the first sound beam and the second sound beam, as well as the at least two pressure sensors may be included in a system. Alternatively, in some embodiments, a transducer configured to propagate the first sound beam may be included in a first system, a transducer configured to propagate the second sound beam may be included in a second system, and the at least two sensors may be included in a third system. In some embodiments, the first system and the third system may be combined. In some embodiments, processing of the signals may occur at the first system and/or at the third system. Note further, that other configurations are also possible.

As a further example, in some embodiments, a first sound beam and a second sound beam may be propagated through an acoustic medium and signals representative of sound pressure in the acoustic medium may be received from at least one sensor. The signals may be processed to determine properties of an interacting region of the first sound beam and the second sound beam, including determining a location of an intersection of the first sound beam and the second sound beam in the acoustic medium, e.g., based on directions of the first sound beam and second sound beam. In some embodiments, the first sound beam may be propagated in a first direction from a first location and a second sound beam may be propagated in a second direction from a second location. In some embodiments, to determine a direction of the first sound beam, a direction and/or focal length of the first sound beam may be adjusted to maximize an amplitude of signals created (e.g., generated) from nonlinear mixing of the first sound beam and the second sound beam. Note that in some embodiments, transducers configured to propagate the first sound beam and the second sound beam, as well as the at least one pressure sensor may be included in a system. Alternatively, in some embodiments, a transducer configured to propagate the first sound beam may be included in a first system, a transducer configured to propagate the second sound beam may be included in a second system, and the at least one sensor may be included in a third system. In some embodiments, the first system and the third system may be combined. In some embodiments, processing of the signals may occur at the first system and/or at the third system. Note further, that other configurations are also possible.

This Summary is intended to provide a brief overview of some of the subject matter described in this document. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF THE DRAWINGS

The following detailed description makes reference to the accompanying drawings, which are now briefly described.

FIG. 1 illustrates an example of a brain ultrasound using an unfocused probe beam, according to some embodiments.

FIGS. 2A and 2B illustrate an example of a computer simulation via finite element analysis of an ultrasound beam propagating and focusing in a human head, according to some embodiments.

FIG. 3A illustrates an example of a computer simulation via finite element analysis of multiple ultrasound beams propagating and focusing in a human head, according to some embodiments.

FIG. 3B illustrates signal spectra from each sensor during the example of the computer simulation via finite element analysis of multiple ultrasound beams propagating and focusing in the human head illustrated in FIG. 3A, according to some embodiments.

FIG. 4 illustrates an example of a brain ultrasound using a focused probe beam, according to some embodiments.

FIG. 5 illustrates an example of locating a focal point of a sound beam, according to some embodiments.

FIG. 6 illustrates an example block diagram of a computer system, according to some embodiments.

FIGS. 7-10 illustrate block diagrams of examples of methods for sound beam focal point determination in an acoustic medium, according to some embodiments.

While the invention is susceptible to various modifications and alternative forms, specific embodiments thereof are shown by way of example in the drawings and are herein described in detail. It should be understood, however, that the drawings and detailed description thereto are not intended to limit the invention to the particular form disclosed, but on the contrary, the intention is to cover all modifications, equivalents and alternatives falling within the spirit and scope of the present invention as defined by the appended claims.

### DETAILED DESCRIPTION OF THE INVENTION

### Terms

The following is a glossary of terms used in this disclosure:

Memory Medium—Any of various types of non-transitory memory devices or storage devices. The term “memory medium” is intended to include an installation medium, e.g., a CD-ROM, floppy disks, or tape device; a computer system memory or random access memory such as DRAM, DDR RAM, SRAM, EDO RAM, Rambus RAM, etc.; a non-volatile memory such as a Flash, magnetic media, e.g., a hard drive, or optical storage; registers, or other similar types of memory elements, etc. The memory medium may include other types of non-transitory memory as well or combinations thereof. In addition, the memory medium may be located in a first computer system in which the programs are executed, or may be located in a second different computer system which connects to the first computer system over a network, such as the Internet. In the latter instance, the second computer system may provide program instructions to the first computer for execution. The term “memory medium” may include two or more memory mediums which may reside in different locations, e.g., in different computer systems that are connected over a network. The memory medium may store program instructions (e.g., embodied as computer programs) that may be executed by one or more processors.

Carrier Medium—a memory medium as described above, as well as a physical transmission medium, such as a bus, network, and/or other physical transmission medium that conveys signals such as electrical, electromagnetic, or digital signals.

Functional Unit (or Processing Element/Processor)—refers to various elements or combinations of elements. Processing elements include, for example, circuits such as an ASIC (Application Specific Integrated Circuit), portions or circuits of individual processor cores, entire processor cores, individual processors, programmable hardware devices such as a field programmable gate array (FPGA), and/or larger portions of systems that include multiple processors, as well as any combinations thereof.

Processing Element/Processor (or Functional Unit)—refers to various elements or combinations of elements. Processing elements include, for example, circuits such as an ASIC (Application Specific Integrated Circuit), portions or circuits of individual processor cores, entire processor cores, individual processors, programmable hardware devices such as a field programmable gate array (FPGA), and/or larger portions of systems that include multiple processors.

Programmable Hardware Element—includes various hardware devices comprising multiple programmable function blocks connected via a programmable interconnect. Examples include FPGAs (Field Programmable Gate Arrays), PLDs (Programmable Logic Devices), FPOAs (Field Programmable Object Arrays), and CPLDs (Complex PLDs). The programmable function blocks may range from fine grained (combinatorial logic or look up tables) to coarse grained (arithmetic logic units or processor cores). A programmable hardware element may also be referred to as “reconfigurable logic”.

Computer System (or Computer)—any of various types of computing or processing systems, including a personal computer system (PC), mainframe computer system, workstation, network appliance, Internet appliance, personal digital assistant (PDA), television system, grid computing system, or other device or combinations of devices. In general, the term “computer system” can be broadly defined to encompass any device (or combination of devices) having at least one processor that executes instructions from a memory medium.

Sound/Soundwave—refers to mechanical and/or elastic waves (including surface acoustic waves) in any medium (gas, liquid, solid, gel, soil, and so forth) and/or combination of media, including interfaces between media. Sound may occur at any amplitude and at any frequency, e.g., ranging from infrasound (e.g., sound waves with frequencies below human audibility, generally considered to be under 20 hertz (Hz)) to ultrasound (e.g., sound waves with frequencies above human audibility, generally considered to be above 20 kilohertz (kHz)), as well as all frequencies in between.

Sound Pressure (or Acoustic Pressure)—refers to a local pressure and/or stress deviation from an ambient (quiescent, average, and/or equilibrium) pressure, caused by a sound wave.

Sound Beam—refers to an area through which sound energy emitted from a sound transducer travels. A sound beam may be three dimensional and symmetrical around its central axis. In some instances, e.g., depending on a sound transducer implemented to generate the sound beam, a sound beam may include a converging region and a diverging region. The regions may intersect at a focal point of the sound beam. In other instances, e.g., depending on a sound transducer implemented to generate the sound beam, a sound beam may include a near field (or Fresnel zone) which is cylindrical in shape and a far field (or Fraunhofer zone) which is conical in shape and in which the sound beam diverges. A sound beam may be a continuous wave, single-frequency beam. Additionally, a sound beam may be tone-bursts, e.g., comprised of a finite number of cycles at a particular frequency. Further, a sound beam may be broadband, comprised of a finite duration pulse and having a broad spectral content.

Medium (or Acoustic Medium)—refers to any substance through which sound may pass, including, but not limited to a gas, a liquid, a solid, a gel, soil, and so forth. Example of mediums may include, but are not limited to, various portions of human anatomy, including soft tissue such as muscle (e.g., heart) and organs (e.g., brain, kidney, lungs, and so forth) as well as hard tissue (e.g., such as bones), as well as various layers of the earth, including the crust, the upper mantle, the mantle, the outer core, and the inner core.

Automatically—refers to an action or operation performed by a computer system (e.g., software executed by the computer system) or device (e.g., circuitry, programmable hardware elements, ASICs, etc.), without user input directly specifying or performing the action or operation. Thus, the term “automatically” is in contrast to an operation being manually performed or specified by the user, where the user provides input to directly perform the operation. An automatic procedure may be initiated by input provided by the user, but the subsequent actions that are performed “automatically” are not specified by the user, i.e., are not performed “manually”, where the user specifies each action to perform. For example, a user filling out an electronic form by selecting each field and providing input specifying information (e.g., by typing information, selecting check boxes, radio selections, etc.) is filling out the form manually, even though the computer system must update the form in response to the user actions. The form may be automatically filled out by the computer system where the computer system (e.g., software executing on the computer system) analyzes the fields of the form and fills in the form without any user input specifying the answers to the fields. As indicated above, the user may invoke the automatic filling of the form, but is not involved in the actual filling of the form (e.g., the user is not manually specifying answers to fields but rather they are being automatically completed). The present specification provides various examples of operations being automatically performed in response to actions the user has taken.

Approximately/Substantially—refers to a value that is almost correct or exact. For example, approximately may refer to a value that is within 1 to 10 percent of the exact (or desired) value. It should be noted, however, that the actual threshold value (or tolerance) may be application dependent. For example, in one embodiment, “approximately” may mean within 0.1% of some specified or desired value, while in various other embodiments, the threshold may be, for example, 2%, 3%, 5%, and so forth, as desired or as required by the particular application. Furthermore, the term approximately may be used interchangeable with the term substantially. In other words, the terms approximately and substantially are used synonymously to refer to a value, or shape, that is almost correct or exact.

Couple—refers to the combining of two or more elements or parts. The term “couple” is intended to denote the linking of part A to part B, however, the term “couple” does not exclude the use of intervening parts between part A and part B to achieve the coupling of part A to part B. For example, the phrase “part A may be coupled to part B” means that part A and part B may be linked indirectly, e.g., via part C. Thus, part A may be connected to part C and part C may be connected to part B to achieve the coupling of part A to part B.

The headings used herein are for organizational purposes only and are not meant to be used to limit the scope of the description. As used throughout this application, the word “may” is used in a permissive sense (i.e., meaning having the potential to), rather than the mandatory sense (i.e., meaning must). The words “include,” “including,” and “includes” indicate open-ended relationships and therefore mean including, but not limited to. Similarly, the words “have,” “having,” and “has” also indicated open-ended relationships, and thus mean having, but not limited to. The terms “first,” “second,” “third,” and so forth as used herein are used as labels for nouns that they precede, and do not imply any type of ordering (e.g., spatial, temporal, logical, etc.) unless such an ordering is otherwise explicitly indicated. For example, a “third component electrically connected to the module substrate” does not preclude scenarios in which a “fourth component electrically connected to the module substrate” is connected prior to the third component, unless otherwise specified. Similarly, a “second” feature does not require that a “first” feature be implemented prior to the “second” feature, unless otherwise specified.

Various components may be described as “configured to” perform a task or tasks. In such contexts, “configured to” is a broad recitation generally meaning “having structure that” performs the task or tasks during operation. As such, the component can be configured to perform the task even when the component is not currently performing that task (e.g., a set of electrical conductors may be configured to electrically connect a module to another module, even when the two modules are not connected). In some contexts, “configured to” may be a broad recitation of structure generally meaning “having circuitry that” performs the task or tasks during operation. As such, the component can be configured to perform the task even when the component is not currently on. In general, the circuitry that forms the structure corresponding to “configured to” may include hardware circuits.

Various components may be described as performing a task or tasks, for convenience in the description. Such descriptions should be interpreted as including the phrase “configured to.” Reciting a component that is configured to perform one or more tasks is expressly intended not to invoke 35 U.S.C. § 112(f) interpretation for that component.

The scope of the present disclosure includes any feature or combination of features disclosed herein (either explicitly or implicitly), or any generalization thereof, whether or not it mitigates any or all of the problems addressed herein. Accordingly, new claims may be formulated during prosecution of this application (or an application claiming priority thereto) to any such combination of features. In particular, with reference to the appended claims, features from dependent claims may be combined with those of the independent claims and features from respective independent claims may be combined in any appropriate manner and not merely in the specific combinations enumerated in the appended claims.

### Sound Beam Focal Point Determination

In current implementations, high-intensity focused ultrasound (HIFU or FUS) may be used for medical purposes, e.g., such as for the treatment of tumors in soft tissue. In such instances, an intensity of the ultrasound at a focus (e.g., focal point) of a therapeutic beam (e.g., sound beam) propagated through the soft tissue as part of the ultrasound may be large enough to cause coagulative necrosis due to localized heating or liquefaction of tissue via extreme mechanical stresses. Further, because real-time observation of the focal point may be critical to ensuring complete destruction of the tumor (or more generally, target) without damaging adjacent healthy tissue, a number of therapeutic HIFU/FUS systems have been integrated with conventional diagnostic (imaging) ultrasound systems in order to provide “guidance” of the therapeutic beam, resulting in ultrasound guided focused ultrasound (USgFUS). The predominant imaging modality for the diagnostic ultrasound is traditional brightness-mode (B-mode) grayscale imaging, which is able to detect changes in the acoustic properties of the tissue which result from the therapeutic beam. However, further improvements are desired.

Embodiments described herein provide systems, methods, and mechanisms for sound beam focal point determination in a three-dimensional space, e.g., based on nonlinear mixing of sound beams in an acoustic medium. In some embodiments, a second sound beam may be used to locate the focal point of a focused sound beam in three-dimensional space. Sensors may measure sound pressure within the acoustic medium to aid in determination of the focal point location. In some embodiments, the sensors may include any, any combination of, and/or all of microphones, hydrophones, accelerometers, strain gauges, Laser Doppler Velocimeters (LDVs), Laser Doppler Anemometers (LDAs), and/or geophones (e.g., a voice coil in reverse), among other pressure, stress, strain, and/or sound sensors. Note further that the sensors may include systems using laser Doppler and/or optical interference to measure the sound pressure. Signals from one or more sensors may be processed to detect nonlinear mixed signals (e.g., such as sum and/or difference tones) generated by the propagation of multiple beams through the acoustic medium. The nonlinear mixed signals may be used in the determination of the focal point, at least in some embodiments.

For example, a signal beam (e.g., a focused beam) may be propagated in a medium (e.g., an acoustic medium) and a probe beam may be introduced into the medium to cross the signal beam. An intersection of the signal beam and the probe beam may be considered an interacting region and/or region of interest/focus region. In some embodiments, at least one sensor (e.g., one or more and/or a plurality of sensors) may be positioned remotely to measure sound (e.g., sound pressure) in the medium. In response to sensing sound (e.g., sound pressure), the at least one sensor may output a signal(s). The signal(s) from the at least one sensor may be processed (and/or analyzed) to determine properties of the interacting region, e.g., such as local sound pressure as well as three-dimensional location.

As another example, a signal beam (e.g., a focused beam) may be propagated in a medium (e.g., an acoustic medium) with a known direction relative to a coordinate system. The signal beam may be focused to a region of maximum sound pressure and a probe beam may be propagated into the medium with a known direction relative to the coordinate system. In some embodiments, at least one sensor (e.g., one or more and/or a plurality of sensors) may be positioned remotely to measure sound (e.g., sound pressure) in the medium. In response to sensing sound (e.g., sound pressure), the at least one sensor may output a signal(s). The signal(s) from the at least one sensor may be processed (and/or analyzed) to determine when the probe beam illuminates the region of maximum sound pressure of the signal beam. In some embodiments, the known propagation direction of the signal beam and the probe beam may be used to compute location of the maximum sound pressure of the signal beam.

In some embodiments, the signal beam may have energy concentrated at a first frequency and the probe beam may have energy concentrated at a second frequency. A signal processing scheme may be used to detect signal information resulting from nonlinear mixing of the signal beam and the probe beam. For example, the signal processing scheme may be used to detect signal information concentrated at a sum frequency (e.g., a sum of the first frequency and the second frequency). As another example, the signal processing scheme may be used to detect signal information concentrated at a difference frequency (e.g., a difference between the first frequency and the second frequency).

Additionally, the signal processing scheme may be used to determine a location (e.g., coordinates) of the interacting region, a volume of the interacting region, a location of the focal point of the signal beam and/or probe beam, a peak sound pressure of the signal beam and/or a peak sound pressure of the probe beam, and/or a width and/or depth of a focal region of the signal beam and/or of the probe beam. Further, the signal processing scheme may be used to generate an image of the signal beam in the interacting region. In some embodiments, diagnostic ultrasound imaging may be included to determine a location of other objects and/or structures relative to the focus region of the first beam and/or the second beam.

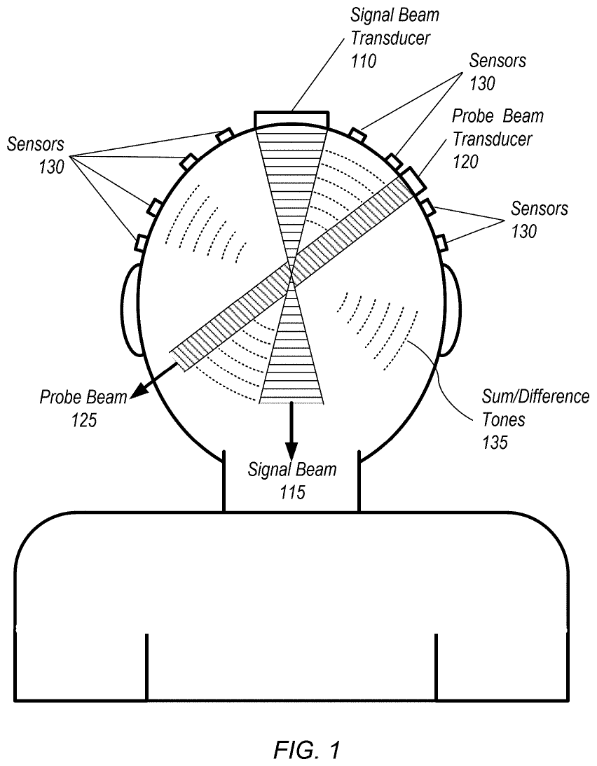

For example, FIG. 1 illustrates an example of a brain ultrasound using an unfocused probe beam, according to some embodiments. As shown, a signal (sound) beam transducer 110 may focus a signal beam 115 with energy concentrated at a first frequency, f₁, into an acoustic medium (e.g., such as a brain) in a first direction. In some embodiments, focusing the signal beam 115 may result in a focal point with high sound intensity (e.g., high sound pressure as compared to sound pressure in adjacent/surrounding medium), which may be used to treat or image a certain region (e.g., region of interest) of the brain, e.g., the primary goal of the brain ultrasound. In addition, a secondary goal of the brain ultrasound may be to accurately (and/or precisely) locate a position of the focal point within the brain, e.g., within some specified tolerance of a target location.

As shown, a probe beam transducer 120 (e.g., such as a phased-array transducer) may introduce a probe beam 125 with energy concentrated at a second frequency, f₂, into the acoustic medium. The probe beam 125 may be unfocused, as shown. In some embodiments, when the probe beam 125 intersects with the signal beam 115 and their amplitudes are large enough to introduce nonlinear effects, the two beams may mix and interact to produce nonlinear mixing of the signal beam 115 and the probe beam 125. As a result of the nonlinear mixing, sound pressure may be at a maximum at a sum frequency, f₁+f₂, and a difference frequency, f₁−f₂. The sum frequency may be referred to as a sum tone (e.g., sum/difference tones 135) and the difference frequency may be referred to as a difference tone (e.g., sum/difference tones 135). In some embodiments, signals generated and/or created from the nonlinear mixing (e.g., such as the sum tone and/or difference tone) may be used to locate a position of the focal point of the signal beam 115 and/or a region of interaction between the signal beam 115 and the probe beam 125.

For example, at least one sensor 130 may be used to detect (e.g., listen) for signals generated (and/or created) from the nonlinear mixing of the signal beam 115 and the probe beam 125. The at least one sensor 130 may measure amplitudes of the signals while the probe beam 125 is steered to scan a region of interest. In some embodiments, a focal point of the signal beam 115 may be identified based on a scan position where amplitudes of the signals are maximized. At this scan position, an axis of the probe beam 125 may intersect an axis of the signal beam 115. For example, based on the scan position and the first direction, a location of the intersection may be determined. In some embodiments, multiple probe beams may be used to enhance accuracy of determining the location of the focal point.

As another example, as shown, two or more sensors 130 may be used to detect (e.g., listen) for signals generated (and/or created) from the nonlinear mixing of the signal beam 125 and the probe beam 115. The two or more sensors 130 may measure amplitudes of the signals and transmit signals representative of the amplitudes to a processing device. The processing device may process and analyze the signals to determine a location of their origin, which may correspond to a focal point of the signal beam 115. In some embodiments, processing may, for example, be based, at least in part, on time-of-arrival analysis and/or any other technique from acoustic array signal processing used to localize a sound source.

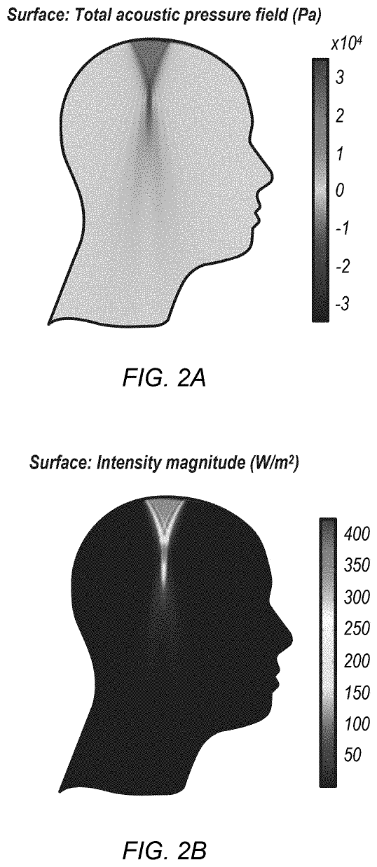

FIGS. 2A and 2B illustrate an example of a computer simulation via finite element analysis of an ultrasound beam propagating and focusing in a human head, according to some embodiments. As shown, the ultrasound beam may be propagated and focused at 500 kilohertz. Further, for the computer simulation, the interior of the human head (e.g., brain) is modeled as water and the skull is not included in the simulation. In particular, FIG. 2A illustrates an acoustic pressure field caused by the ultrasound beam and FIG. 2B illustrates an intensity magnitude of the ultrasound beam. As can be seen, as the ultrasound beam propagates into the human head, the ultrasound beam is focused to a focal point and then begins to disperse.

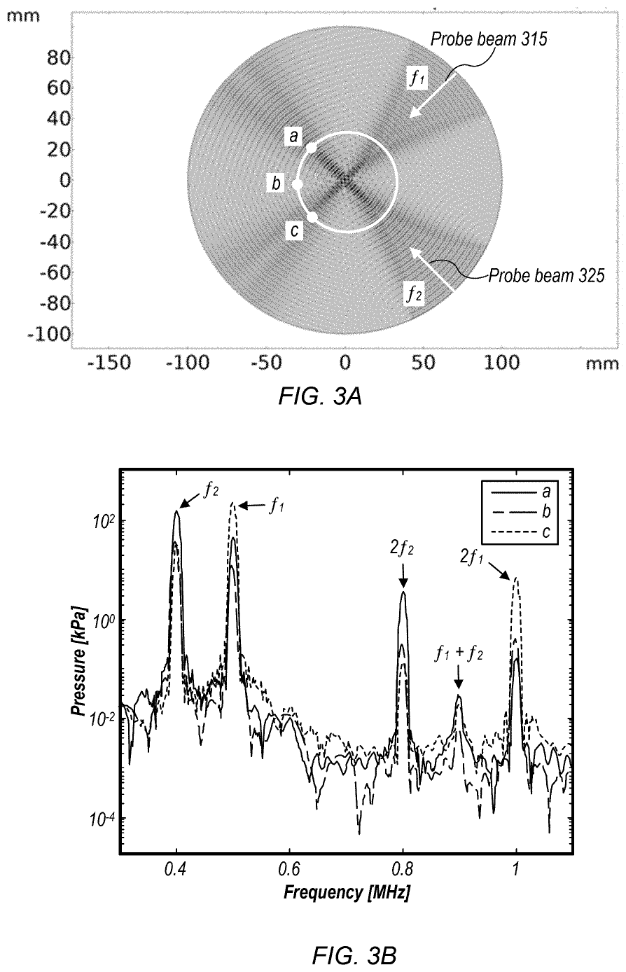

Further, FIG. 3A illustrates an example of a computer simulation via finite element analysis of multiple ultrasound beams propagating and focusing in a human head, according to some embodiments. Note that in the exemplary simulation, losses in the medium are not included. Further, for the computer simulation, the interior of the human head (e.g., brain) is modeled as water and the skull is not included in the simulation. However, inherent nonlinearity in governing momentum and continuity equations from fluid mechanics are retained up to second order, e.g., as is the nonlinear relationship between pressure and density in the medium (e.g., the nonlinearity in the state equation). Furthermore, this particular simulation does not include boundary layer effects as cumulative nonlinear effects are more important than local nonlinear effects. The governing equation used in the finite element analysis is the lossless version of the non-homogeneous Westervelt's equation, as shown by equation 1.

$${∇}^{2} p - \frac{1}{{c}_{o}^{2}} \frac{{\partial }^{2} p}{\partial {t}^{2}} = - \frac{β}{{ρ}_{o} {c}_{o}^{4}} \frac{{\partial }^{2} {p}^{2}}{\partial {t}^{2}} \qquad [ 1 ]$$

Additionally, sensors are included in the simulation, and their positions are shown labeled as “a”, “b”, and “c”. A first probe beam 315 with energy concentrated at a first frequency, f₁, may be propagated in a first direction and a second probe beam 325 with energy concentrated at a second frequency, f₂, may be propagated in a second direction, as shown. The sensors at locations “a”, “b”, and “c” may measure sound pressure in the medium and output various signals. For example, FIG. 3B illustrates signal spectra from each sensor. As shown, peaks at f₁ and f₂ are present, as are harmonics 2f₁ and 2f₂ that are generated due to nonlinearity of the medium. Sum and difference tones at f₁+f₂ and f₁−f₂ are also present due to the nonlinear mixing of the two ultrasound beams, however, in FIG. 3B, only the sum tone appears due to the limited frequency range presented. The sum tone signal is small but observable. In some embodiments, the sum tone signal (and/or the difference tone signal) may be used to determine a location of an intersection of the first probe beam 315 and the second probe beam 325. In some embodiments, the location of the intersection may also correspond to a focal point of the first probe beam 315.

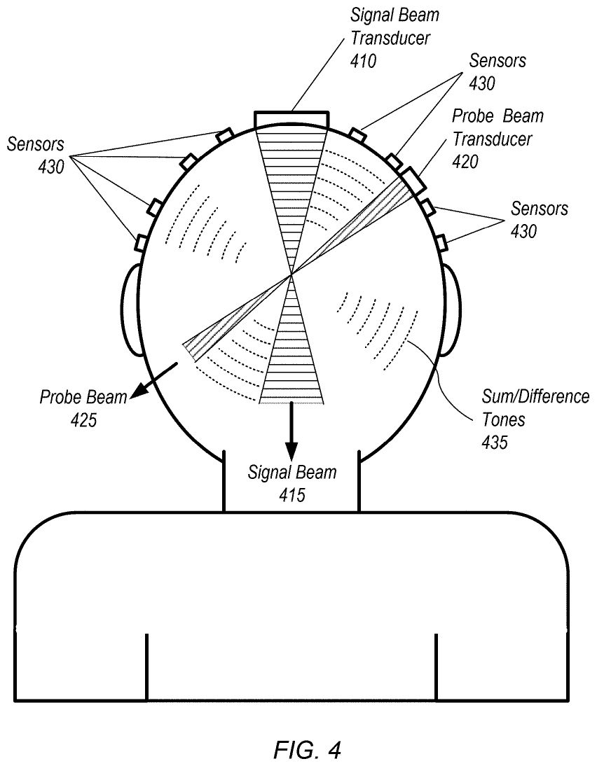

FIG. 4 illustrates an example of a brain ultrasound using a focused probe beam, according to some embodiments. As shown, a signal (sound) beam transducer 410 may focus a signal beam 415 with energy concentrated a first frequency, f₁, into an acoustic medium (e.g., such as a brain) in a first direction. In some embodiments, focusing the signal beam 415 may result in a focal point with high sound intensity (e.g., high sound pressure as compared to sound pressure in adjacent/surrounding medium), which may be used to treat or image a certain region (e.g., region of interest) of the brain, e.g., the primary goal of the brain ultrasound. In addition, a secondary goal of the brain ultrasound may be to accurately (and/or precisely) locate a position of the focal point within the acoustic medium, e.g., within some specified tolerance of a target location.

As shown, a probe beam transducer 420 (e.g., such as a phased-array transducer) may introduce a probe beam 425 with energy concentrated at a second frequency, f₂, into the acoustic medium. The probe beam 425 may be focused, as shown. In some embodiments, a focal point of the probe beam 425 may be moved within the acoustic medium, e.g., to induce an intersection of the focal point of probe beam 425 with the focal point of signal beam 415. For example, a direction, focal length, frequency, amplitude, and/or a focal depth of the probe beam 425 may be adjusted (e.g., in an iterative and/or learning manner) until the intersection is located. In other words, a focal point of the probe beam 425 may be swept through the acoustic medium in order to locate an intersection with the focal point of the signal beam 415. In some embodiments, when the focal point of the probe beam 425 intersects with the focal point of the signal beam 415 and their amplitudes are large enough to introduce nonlinear effects, the two beams may mix and interact to produce nonlinear mixing of the signal beam 415 and the probe beam 425. As a result of the nonlinear mixing, sound pressure and/or sound stress may be at a maximum at a sum frequency, f₁+f₂, and a difference frequency, f₁−f₂. The sum frequency may be referred to as a sum tone (e.g., sum/difference tones 435) and the difference frequency may be referred to as a difference tone (e.g., sum/difference tones 435). In some embodiments, signals generated and/or created from the nonlinear mixing (e.g., such as the sum tone and/or difference tone) may be used to locate a position of the focal point of the signal beam 415 and/or a region of interaction between the signal beam 415 and the probe beam 425. Thus, as further described herein, determination of the intersection may be based, at least in part, on detection of the signals generated and/or created from the nonlinear mixing.

For example, at least one sensor 430 may be used to detect (e.g., listen) for signals generated (and/or created) from the nonlinear mixing of the signal beam 415 and the probe beam 425. The at least one sensor 430 may measure amplitudes of the signals while a focal point of the probe beam 425 is steered (e.g., swept and/or adjusted) to scan a region of interest. In some embodiments, a focal point of the signal beam 415 may be identified based on a scan position where amplitudes of the signals are maximized. At this scan position, an axis of the probe beam 425 may intersect an axis of the signal beam 415. For example, based on the scan position and the first direction, a location of the intersection may be determined. In some embodiments, multiple probe beams may be used to enhance accuracy of determining the location of the focal point.

As another example, as shown, two or more sensors 430 may be used to detect (e.g., listen) for signals generated (and/or created) from the nonlinear mixing of the signal beam 415 and the probe beam 425. The two or more sensors 430 may measure amplitudes of the signals while a focal point of the probe beam 425 is steered (e.g., swept and/or adjusted). The two or more sensors 430 may transmit signals representative of the amplitudes to a processing device. The processing device may process and analyze the signals to determine a location of their origin when the amplitudes of the signals are maximized, which may correspond to a focal point of the signal beam 415. In some embodiments, processing may, for example, be based, at least in part, on time-of-arrival analysis and/or any other technique from acoustic array signal processing used to localize a sound source.

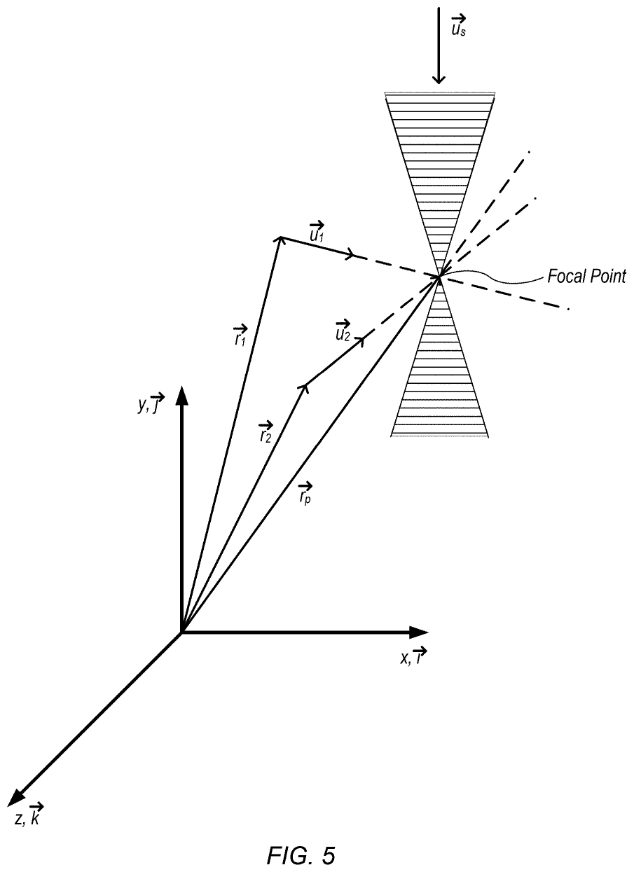

FIG. 5 illustrates an example of locating a focal point of a sound beam in a three-dimensional space, according to some embodiments. As shown, a reference frame may include three orthogonal axes (e.g., x, y, and z). Additionally, a propagation direction of a focused sound beam may be given by a vector, \vec{u}ₛ, and the focused sound beam may have a focal point as shown. A first position vector, \vec{r}₁, may identify a known position of a first source generating a first probe beam. Similarly, a second position vector, \vec{r}²₂, may identify a known position of a second source generating a second probe beam. Further, as shown, a first directional unit vector, \vec{u}₁, may point from the first source at \vec{r}₁ to the focal point. Note that this direction may be discoverable via measurement, e.g., by turning off the second probe beam and adjusting a direction of the first probe beam until signals generated (and/or created) from the nonlinear mixing of the beams (e.g., sum and difference tones) are detected with maximum strength. Similarly, as shown, a second directional vector, \vec{u}₂, may point from the second source at \vec{r}₂ to the focal point. Note that this direction may be discoverable via measurement, e.g., by turning off the first probe beam and adjusting a direction of the second probe beam until signals generated (and/or created) from the nonlinear mixing of the beams (e.g., sum and difference tones) are detected with maximum strength. Additionally, knowledge of \vec{r}₁, \vec{u}₁, \vec{r}₂ and \vec{u}₂ may enable the position of the focal point \vec{r}ₚ to be determined. For example, vector arithmetic gives the expression as shown in equation 2, e.g.:

\vec{r}_{f}=\vec{r}₁+a\vec{u}₁=\vec{r}₂+b\vec{u}₂ [2]

where a is a distance from the first source at \vec{r}₁ to the focal point, and b is a distance from the second source at \vec{r}₂ to the focal point. The vector expression may be solved for the scalars a and b, and the coordinates of the focal point thus determined.

Note that although in the example of FIG. 5 two probe beams are used, only one probe beam is required to determine location of the focal point. As such, when two or more probe beams are used, redundant information may be provided and may be used to increase accuracy of determination of the focal point location.

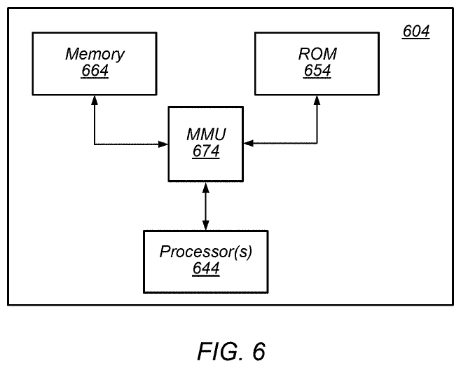

FIG. 6 illustrates an example block diagram of a computer system 604, according to some embodiments. It is noted that the computer system of FIG. 6 is merely one example of a possible computer system. As shown, the computer system 604 may include processor(s) 644 which may execute program instructions for the computer system 604. The processor(s) 644 may also be coupled to memory management unit (MMU) 674, which may be configured to receive addresses from the processor(s) 644 and translate those addresses to locations in memory (e.g., memory 664 and read only memory (ROM) 654) or to other circuits or devices.

The computer system 604 may include hardware and software components for implementing or supporting implementation of features described herein. The processor 644 of the computer system 604 may be configured to implement or support implementation of part or all of the methods described herein, e.g., by executing program instructions stored on a memory medium (e.g., a non-transitory computer-readable memory medium). Alternatively, the processor 644 may be configured as a programmable hardware element, such as an FPGA (Field Programmable Gate Array), or as an ASIC (Application Specific Integrated Circuit), or a combination thereof. Alternatively (or in addition) the processor 644 of the computer system 604, in conjunction with one or more of the other components 654, 664, and/or 674 may be configured to implement or support implementation of part or all of the features described herein.

In addition, as described herein, processor(s) 644 may be comprised of one or more processing elements. In other words, one or more processing elements may be included in processor(s) 644. Thus, processor(s) 644 may include one or more integrated circuits (ICs) that are configured to perform the functions of processor(s) 644. In addition, each integrated circuit may include circuitry (e.g., first circuitry, second circuitry, etc.) configured to perform the functions of processor(s) 644.

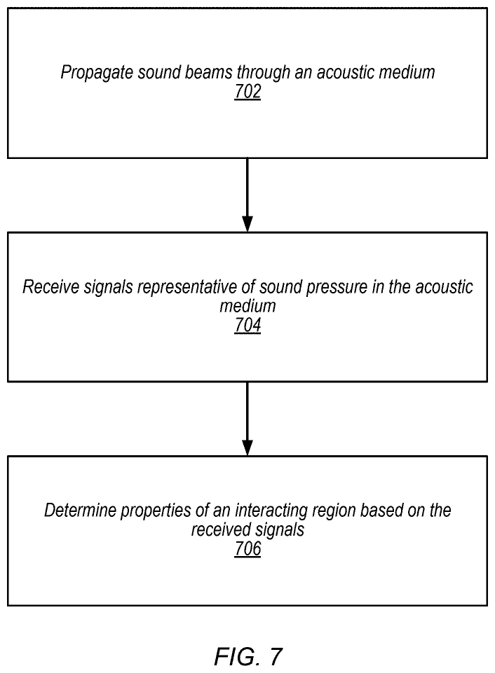

FIG. 7 illustrates a block diagram of an example of a method for sound beam focal point determination in an acoustic medium, according to some embodiments. The method shown in FIG. 7 may be used in conjunction with any of the systems, methods, or devices shown in the Figures, among other devices. In various embodiments, some of the method elements shown may be performed concurrently, in a different order than shown, or may be omitted. Additional method elements may also be performed as desired. As shown, this method may operate as follows.

At 702, sound beams (e.g., a first sound beam, such as a probe beam, and a second sound beam, such as a signal beam) may be propagated through an acoustic medium. In some embodiments, the sound beams may be generated by respective probe beam transducers (e.g., such as a phased-array transducer). In some embodiments, a first controller and/or control system (e.g., computer system) may generate the first sound beam (e.g., a probe beam) and a second controller and/or control system may generate the second sound beam (e.g., a signal beam) via the respective probe beam transducers. In other words, a first system may control propagation of the first sound beam and a second system may control propagation of the second sound beam. Alternatively, in some embodiments, the first controller and/or control system (e.g., computer system) may generate the first sound beam (e.g., a probe beam) and the second sound beam (e.g., a signal beam). In some embodiments, the first sound beam may be intersected by the second sound beam, e.g., in an interacting region. The first sound beam may be propagated with energy concentrated at a first frequency and the second sound beam may be propagated with energy concentrated at a second frequency. In some embodiments, the first frequency and the second frequency may be different. In some embodiments, the first sound beam may be (and/or be considered) a probe beam. In some embodiments, the second sound beam may be (and/or may be considered) a signal beam. In some embodiments, the signal beam may be a focused beam, e.g., the signal beam may have (and/or converge at) a focal point. In some embodiments, the probe beam may be focused or unfocused, e.g., the probe beam may or may not have (and/or may or may not converge at) a focal point.

At 704, signals representative of sound pressure and/or sound stress in the acoustic medium may be received, e.g., from one or more (pressure and/or stress/strain) sensors. In some embodiments, the signals may be received by a signal processing system. In some embodiments, the signal processing system may be included in the first controller and/or control system generating the first sound beam. Alternatively, when the second controller and/or control system generates the first sound beam and the second sound beam, the signal processing system may be included in the second controller and/or control system.

At 706, properties of an interacting region of the sound beams may be determined based on the received signals. In other words, the received signals may be processed to determine the properties of the interacting region. In some embodiments, signals generated from nonlinear mixing of the first sound beam and second sound beam may be used to determine properties of the interacting region. For example, signals concentrated at a sum of the first frequency and the second frequency (e.g., a sum tone) and/or signals concentrated at a difference of the first frequency and the second frequency (e.g., a difference tone) may be used to determine properties of the interacting region. In some embodiments, properties of the interacting region may include a location of an intersection of the first sound beam and the second sound beam in the acoustic medium. In some embodiments, the location may be relative to a reference location. In some embodiments, the location may be specified in three-dimensional space relative to a reference frame. In some embodiments, properties of the interacting region may include any, any combination of, and/or all of (e.g., at least one of and/or one or more of) a volume of the interacting region, a peak sound pressure of the first sound beam, a peak sound pressure of the second sound beam, a width and depth of a focal region of the first sound beam, and/or a width and depth of a focal region of the second sound beam. In some embodiments, processing the signals to determine properties of the interacting region may include processing the signals to generate an image of one of the first sound beam or second sound beam in the interacting region.

In some embodiments, signals from an ultrasound probe may be received, e.g., by the first controller and/or control system, to determine location of one or more objects and/or structures in the acoustic medium. In some embodiments, the location may be relative to a focal point or focal region of one of the first sound beam or second sound beam.

In some embodiments, the first sound beam may include a first focal point at a first location within the acoustic medium and the second sound beam may include (and/or converge at) a second focal point. In such embodiments, a location of the first focal point within the acoustic medium may be adjusted, e.g., based at least in part, on the received signals. For example, adjusting the location of the first focal point (e.g., sweeping and/or moving the location of the first focal point) within the acoustic medium may include any, any combination of, and/or all of (e.g., at least one of and/or one or more of) adjusting a direction of the first sound beam, adjusting a frequency of the first sound beam, adjusting an amplitude of the first sound beam, adjusting a depth of the first focal point, and/or adjusting a focal length of the first sound beam. In some embodiments, signals generated from the nonlinear mixing of the first sound beam and the second sound beam as the location of the first focal point is adjusted may be detected. In some embodiments, the second location may be determined based on detection of a maximum amplitude of signals generated from the nonlinear mixing of the first sound beam and the second sound beam. In some embodiments, detection of the maximum amplitude may include receiving signals representative of sound pressure and/or sound stress in the acoustic medium from one or more pressure (and/or stress/strain) sensors. In some embodiments, the location of the second focal point may be determined based, at least in part, on time-of-arrival analysis of the received signals representative of sound pressure and/or sound stress in the acoustic medium from the one or more pressure (and/or stress/strain) sensors. In some embodiments, the one or more pressure (and/or stress/strain) sensors may be positioned on an external surface of the acoustic medium.

In some embodiments, a first position within a reference frame may correspond to a first source generating the first sound beam and a second position within the reference frame may correspond to a second source generating the second sound beam. In such embodiments, to determine a direction of the first sound beam, a direction of the first sound beam may be adjusted to maximize an amplitude of signals generated from the nonlinear mixing of the first sound beam and the second sound beam. In some embodiments, a position and/or location of a focal point of the second sound beam may be determined based, at least in part, on the first position and direction of the first sound beam.

In some embodiments, an additional (e.g., third) sound beam (e.g., a second probe beam) may be propagated through the acoustic medium, e.g., via an additional (e.g., third) probe beam transducer, e.g., controlled by the first controller and/or control system. In some embodiments, a third position within the reference frame may correspond to a third source generating a third sound beam, where during adjustment of the direction of the first sound beam the third sound beam may not be propagated. In such embodiments, to determine a direction of the third sound beam, a direction of the third sound beam may be adjusted to maximize an amplitude of signals generated from the nonlinear mixing of the second sound beam and the third sound beam, where during adjustment of the direction of the third sound beam the first sound beam may not be propagated. Further, in some embodiments, a position of the focal point of the second sound beam may be determined based, at least in part, on the first position, the third position, direction of the first sound beam, and direction of the third sound beam.

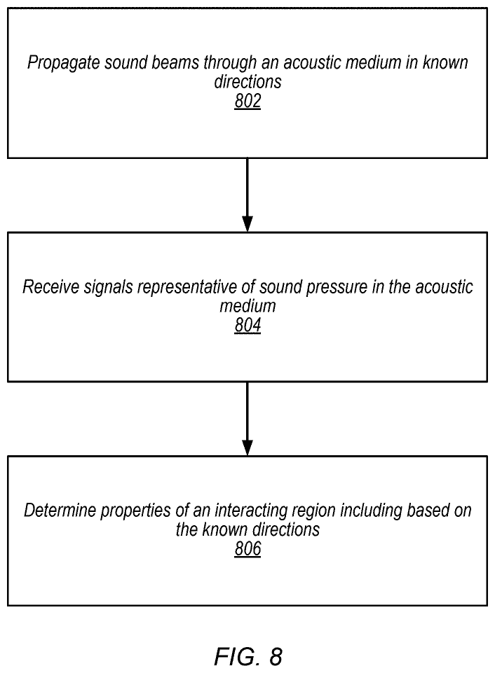

FIG. 8 illustrates a block diagram of an example of a method for sound beam focal point determination in an acoustic medium, according to some embodiments. The method shown in FIG. 8 may be used in conjunction with any of the systems, methods, or devices shown in the Figures, among other devices. In various embodiments, some of the method elements shown may be performed concurrently, in a different order than shown, or may be omitted. Additional method elements may also be performed as desired. As shown, this method may operate as follows.

At 802, sound beams (e.g., a first sound beam, such as a probe beam, and a second sound beam, such as a signal beam) may be propagated through an acoustic medium in known directions. For example, a first sound beam may be propagated in a first direction from a first location and a second sound beam may be propagated in a second direction from a second location. In some embodiments, the first location may be within a reference frame and may correspond to a first source generating the first sound beam. In some embodiments, the second location may be within the reference frame and may correspond to a second source generating the second sound beam. In some embodiments, the sound beams may be generated by respective probe beam transducers (e.g., such as a phased-array transducer). In some embodiments, a first controller and/or control system (e.g., computer system) may generate the first sound beam (e.g., a probe beam) and a second controller and/or control system may generate the second sound beam (e.g., a signal beam) via the respective probe beam transducers. In other words, a first system may control propagation of the first sound beam and a second system may control propagation of the second sound beam. Alternatively, in some embodiments, the first controller and/or control system (e.g., computer system) may generate the first sound beam (e.g., a probe beam) and the second sound beam (e.g., a signal beam). In some embodiments, the first sound beam may be intersected by the second sound beam, e.g., in an interacting region. In some embodiments, the first sound beam may be propagated with energy focused (e.g., concentrated) at a first frequency and the second sound beam may be propagated with energy focused (e.g., concentrated) at a second frequency. In some embodiments, the first frequency and the second frequency may be different. In some embodiments, the first sound beam may be (and/or be considered) a probe beam. In some embodiments, the second sound beam may be (and/or may be considered) a signal beam. In some embodiments, the signal beam may be a focused beam, e.g., the signal beam may have (and/or converge at) a focal point. In some embodiments, the probe beam may be focused or unfocused, e.g., the probe beam may or may not have (and/or may or may not converge at) a focal point.

At 804, signals representative of sound pressure and/or sound stress in the acoustic medium may be received, e.g., from one or more (pressure and/or stress/strain) sensors. In some embodiments, the signals may be received by a signal processing system. In some embodiments, the signal processing system may be included in the first controller and/or control system generating the first sound beam. Alternatively, when the second controller and/or control system generates the first sound beam and the second sound beam, the signal processing system may be included in the second controller and/or control system.

At 806, properties of an interacting region of the sound beams may be determined based on the received signals, e.g., based, at least in part, on the first direction and the second direction. In other words, the received signals may be processed to determine the properties of the interacting region. In some embodiments, signals generated from nonlinear mixing of the first sound beam and second sound beam may be used to determine properties of the interacting region. For example, signals concentrated at a sum of the first frequency and the second frequency (e.g., a sum tone) and/or signals concentrated at a difference of the first frequency and the second frequency (e.g., a difference tone) may be used to determine properties of the interacting region. In some embodiments, properties of the interacting region may include a location of an intersection of the first sound beam and the second sound beam in the acoustic medium. In some embodiments, the location may be relative to a reference location. In some embodiments, the location may be specified in three-dimensional space relative to a reference frame. In some embodiments, properties of the interacting region may include any, any combination of, and/or all of (e.g., at least one of and/or one or more of) a volume of the interacting region, a peak sound pressure of the first sound beam, a peak sound pressure of the second sound beam, a width and depth of a focal region of the first sound beam, and/or a width and depth of a focal region of the second sound beam. In some embodiments, processing the signals to determine properties of the interacting region may include processing the signals to generate an image of one of the first sound beam or second sound beam in the interacting region.

In some embodiments, the first location within the reference frame may correspond to a first source generating the first sound beam and the second location within the reference frame may correspond to a second source generating the second sound beam. In such embodiments, to determine a direction of the first sound beam, a direction of the first sound beam may be adjusted to maximize an amplitude of signals generated from the nonlinear mixing of the first sound beam and the second sound beam. In some embodiments, a position and/or location of a focal point of the second sound beam may be determined based, at least in part, on the first location and direction of the first sound beam.

In some embodiments, signals from an ultrasound probe may be received, e.g., by the first controller and/or control system, to determine location of one or more objects and/or structures in the acoustic medium. In some embodiments, the location may be relative to a focal point or focal region of one of the first sound beam or second sound beam.

In some embodiments, the first sound beam may include a first focal point at a first location within the acoustic medium and the second sound beam may include (and/or converge at) a second focal point. In such embodiments, a location of the first focal point within the acoustic medium may be adjusted, e.g., based at least in part, on the received signals. For example, adjusting the location of the first focal point (e.g., sweeping and/or moving the location of the second focal point) within the acoustic medium may include any, any combination of, and/or all of (e.g., at least one of and/or one or more of) adjusting a direction of the first sound beam, adjusting a frequency of the first sound beam, adjusting an amplitude of the first sound beam, adjusting a depth of the second focal point, and/or adjusting a focal length of the first sound beam. In some embodiments, signals generated from the nonlinear mixing of the first sound beam and the second sound beam as the location of the first focal point is adjusted may be detected. In some embodiments, the first location may be determined based on detection of a maximum amplitude of signals generated from the nonlinear mixing of the first sound beam and the first sound beam. In some embodiments, detection of the maximum amplitude may include receiving signals representative of sound pressure and/or sound stress in the acoustic medium from one or more pressure (and/or stress/strain) sensors. In some embodiments, the location of the second focal point may be determined based, at least in part, on time-of-arrival analysis of the received signals representative of sound pressure and/or sound stress in the acoustic medium from the one or more pressure (and/or stress/strain) sensors. In some embodiments, the one or more pressure (and/or stress/strain) sensors may be positioned on an external surface of the acoustic medium.

In some embodiments, an additional (e.g., third) sound beam (e.g., a second probe beam) may be propagated through the acoustic medium, e.g., via an additional (e.g., third) probe beam transducer, e.g., controlled by the first controller and/or control system. In some embodiments, a third location within the reference frame may correspond to a third source generating a third sound beam, where during adjustment of the direction of the first sound beam the third sound beam may not be propagated. In such embodiments, to determine a direction of the third sound beam, a direction of the third sound beam may be adjusted to maximize an amplitude of signals generated from the nonlinear mixing of the second sound beam and the third sound beam, where during adjustment of the direction of the third sound beam the first sound beam may not be propagated. Further, in some embodiments, a position of the focal point of the second sound beam may be determined based, at least in part, on the first position, the third position, direction of the first sound beam, and direction of the third sound beam.

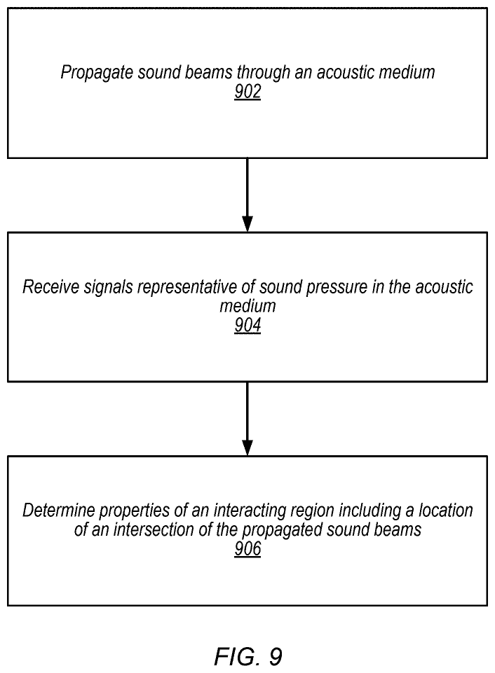

FIG. 9 illustrates a block diagram of an example of a method for sound beam focal point determination in an acoustic medium, according to some embodiments. The method shown in FIG. 9 may be used in conjunction with any of the systems, methods, or devices shown in the Figures, among other devices. In various embodiments, some of the method elements shown may be performed concurrently, in a different order than shown, or may be omitted. Additional method elements may also be performed as desired. As shown, this method may operate as follows.

At 902, sound beams (e.g., a first sound beam, such as a probe beam, and a second sound beam, such as a signal beam) may be propagated through an acoustic medium. In some embodiments, the sound beams may be generated by respective probe beam transducers. In some embodiments, a first controller and/or control system (e.g., computer system) may generate the first sound beam (e.g., a probe beam) and a second controller and/or control system may generate the second sound beam (e.g., a signal beam) via the respective probe beam transducers (e.g., such as a phased-array transducer). In other words, a first system may control propagation of the first sound beam and a second system may control propagation of the second sound beam. Alternatively, in some embodiments, the first controller and/or control system (e.g., computer system) may generate the first sound beam (e.g., a probe beam) and the second sound beam (e.g., a signal beam). In some embodiments, the first sound beam may be intersected by the second sound beam, e.g., in an interacting region. In some embodiments, the first sound beam may be propagated with energy concentrated (e.g., focused) at a first frequency and the second sound beam may be propagated with energy concentrated (e.g., focused) at a second frequency. In some embodiments, the first frequency and the second frequency may be different. In some embodiments, the first sound beam may be (and/or be considered) a probe beam. In some embodiments, the second sound beam may be (and/or may be considered) a signal beam. In some embodiments, the signal beam may be a focused beam, e.g., the signal beam may have (and/or converge at) a focal point. In some embodiments, the probe beam may be focused or unfocused, e.g., the probe beam may or may not have (and/or may or may not converge at) a focal point.

At 904, signals representative of sound pressure and/or sound stress in the acoustic medium may be received, e.g., from two or more (pressure and/or stress/strain) sensors (e.g., a plurality of sensors). In some embodiments, the signals may be received by a signal processing system. In some embodiments, the signal processing system may be included in the first controller and/or control system generating the first sound beam. Alternatively, when the second controller and/or control system generates the first sound beam and the second sound beam, the signal processing system may be included in the second controller and/or control system.

At 906, properties of an interacting region of the sound beams may be determined based on the received signals, e.g., including an intersection of the first sound beam and the second sound beam in the acoustic medium. In other words, the received signals may be processed to determine the properties of the interacting region. In some embodiments, signals generated from nonlinear mixing of the first sound beam and second sound beam may be used to determine properties of the interacting region. For example, signals concentrated at a sum of the first frequency and the second frequency (e.g., a sum tone) and/or signals concentrated at a difference of the first frequency and the second frequency (e.g., a difference tone) may be used to determine properties of the interacting region. In some embodiments, properties of the interacting region may include a location of an intersection of the first sound beam and the second sound beam in the acoustic medium. In some embodiments, the location may be relative to a reference location. In some embodiments, the location may be specified in three-dimensional space relative to a reference frame. In some embodiments, properties of the interacting region may include any, any combination of, and/or all of (e.g., at least one of and/or one or more of) a volume of the interacting region, a peak sound pressure of the first sound beam, a peak sound pressure of the second sound beam, a width and depth of a focal region of the first sound beam, and/or a width and depth of a focal region of the second sound beam. In some embodiments, processing the signals to determine properties of the interacting region may include processing the signals to generate an image of one of the first sound beam or second sound beam in the interacting region.

In some embodiments, the first sound beam may include a first focal point at a first location within the acoustic medium and the second sound beam may include (and/or converge at) a second focal point. In such embodiments, a location of the first focal point within the acoustic medium may be adjusted, e.g., based at least in part, on the received signals. For example, adjusting the location of the first focal point (e.g., sweeping and/or moving the location of the first focal point) within the acoustic medium may include any, any combination of, and/or all of (e.g., at least one of and/or one or more of) adjusting a direction of the first sound beam, adjusting a frequency of the first sound beam, adjusting an amplitude of the first sound beam, adjusting a depth of the first focal point, and/or adjusting a focal length of the first sound beam. In some embodiments, signals generated from the nonlinear mixing of the first sound beam and the second sound beam as the location of the first focal point is adjusted may be detected. In some embodiments, the first location may be determined based on detection of a maximum amplitude of signals generated from the nonlinear mixing of the first sound beam and the second sound beam. In some embodiments, detection of the maximum amplitude may include receiving signals representative of sound pressure and/or sound stress in the acoustic medium from the two or more pressure (and/or stress/strain) sensors. In some embodiments, the location of the second focal point may be determined based, at least in part, on time-of-arrival analysis of the received signals representative of sound pressure and/or sound stress in the acoustic medium from the one or more pressure (and/or stress/strain) sensors. In some embodiments, the two or more pressure (and/or stress/strain) sensors may be positioned on an external surface of the acoustic medium.

In some embodiments, signals from an ultrasound probe may be received, e.g., by the first controller and/or control system, to determine location of one or more objects and/or structures in the acoustic medium. In some embodiments, the location may be relative to a focal point or focal region of one of the first sound beam or second sound beam.

In some embodiments, a first position within a reference frame may correspond to a first source generating the first sound beam and a second position within the reference frame may correspond to a second source generating the second sound beam. In such embodiments, to determine a direction of the first sound beam, a direction of the first sound beam may be adjusted to maximize an amplitude of signals generated from the nonlinear mixing of the first sound beam and the second sound beam. In some embodiments, a position and/or location of a focal point of the second sound beam may be determined based, at least in part, on the first position and direction of the first sound beam.

In some embodiments, an additional (e.g., third) sound beam (e.g., a second probe beam) may be propagated through the acoustic medium, e.g., via an additional (e.g., third) probe beam transducer, e.g., controlled by the first controller and/or control system. In some embodiments, a third position within the reference frame may correspond to a third source generating a third sound beam, where during adjustment of the direction of the first sound beam the third sound beam may not be propagated. In such embodiments, to determine a direction of the third sound beam, a direction of the third sound beam may be adjusted to maximize an amplitude of signals generated from the nonlinear mixing of the second sound beam and the third sound beam, where during adjustment of the direction of the third sound beam the first sound beam may not be propagated. Further, in some embodiments, a position of the focal point of the second sound beam may be determined based, at least in part, on the first position, the third position, direction of the first sound beam, and direction of the third sound beam.

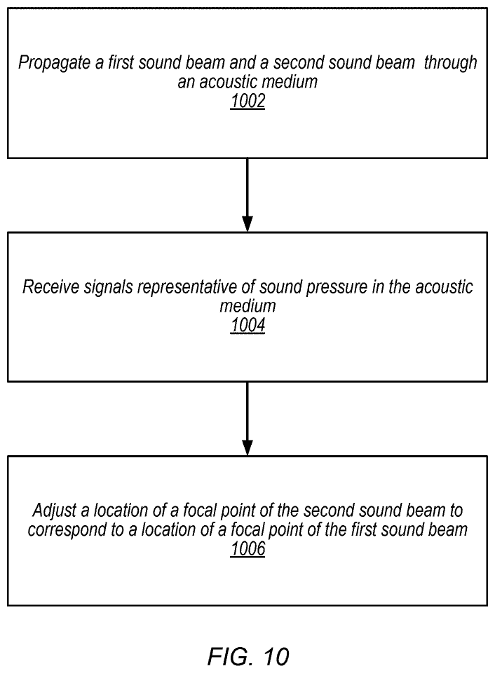

FIG. 10 illustrates a block diagram of an example of a method for sound beam focal point determination in an acoustic medium, according to some embodiments. The method shown in FIG. 10 may be used in conjunction with any of the systems, methods, or devices shown in the Figures, among other devices. In various embodiments, some of the method elements shown may be performed concurrently, in a different order than shown, or may be omitted. Additional method elements may also be performed as desired. As shown, this method may operate as follows.

At 1002, a first sound beam and a second sound beam may be propagated through an acoustic medium. In some embodiments, the sound beams may be generated by respective probe beam transducers (e.g., such as a phased-array transducer). In some embodiments, a first controller and/or control system (e.g., computer system) may generate the first sound beam (e.g., a probe beam) and a second controller and/or control system may generate the second sound beam (e.g., a signal beam) via the respective probe beam transducers. In other words, a first system may control propagation of the first sound beam and a second system may control propagation of the second sound beam. Alternatively, in some embodiments, the first and/or second controller and/or control system (e.g., computer system) may generate the first sound beam (e.g., a signal beam) and the second sound beam. In some embodiments, the first sound beam may be intersected by the second sound beam, e.g., in an interacting region. In some embodiments, the first sound beam may be propagated with energy concentrated (and/or focused) at a first frequency and the second sound beam may be propagated with energy concentrated (and/or focused) at a second frequency. In some embodiments, the first frequency and the second frequency may be different. In some embodiments, the first sound beam may be (and/or be considered) a probe beam. In some embodiments, the second sound beam may be (and/or may be considered) a signal beam. In some embodiments, the signal beam may be a focused beam, e.g., the signal beam may have (and/or converge at) a second focal point. In some embodiments, the probe beam may be focused, e.g., the probe beam may converge at a first focal point.

At 1004, signals representative of sound pressure and/or sound stress in the acoustic medium may be received, e.g., from two or more (pressure and/or stress/strain) sensors (e.g., a plurality of sensors). In some embodiments, the signals may be received by a signal processing system. In some embodiments, the signal processing system may be included in the second controller and/or control system generating the second sound beam. Alternatively, when the first and/or second controller and/or control system generates the first sound beam and the second sound beam, the signal processing system may be included in the first and/or second controller and/or control system.

At 1006, a location of the first focal point within the acoustic medium may be adjusted, e.g., based at least in part, on the received signals, to correspond to a location of the second focal point. In some embodiments, the location of the first focal point may be adjusted to maximize signals generated from nonlinear mixing of the first sound beam and the second sound beam. In some embodiments, the location of the first focal point, corresponding to the maximum amplitude of signals generated from the nonlinear mixing of first sound beam and the second sound beam, may be determined by performing a time-of-arrival analysis on the received signals, e.g., on soundwaves (and/or sound pressure) detected in the acoustic medium via the two or more sensors. In some embodiments, the two or more sensors may be positioned on an external surface of the acoustic medium.

In some embodiments, adjusting the location of the first focal point (e.g., sweeping and/or moving the location of the first focal point) within the acoustic medium may include any, any combination of, and/or all of (e.g., at least one of and/or one or more of) adjusting a direction of the first sound beam, adjusting a frequency of the first sound beam, adjusting an amplitude of the first sound beam, adjusting a depth of the first focal point, and/or adjusting a focal length of the first sound beam.

In some embodiments, in addition to determining the intersection, the received signals may be processed to determine properties of an interacting region of the first sound beam and the second sound beam. In some embodiments, signals generated from nonlinear mixing of the first sound beam and second sound beam may be used to determine properties of the interacting region. For example, signals concentrated at a sum of the first frequency and the second frequency (e.g., a sum tone) and/or signals concentrated at a difference of the first frequency and the second frequency (e.g., a difference tone) may be used to determine properties of the interacting region. In some embodiments, properties of the interacting region may include a location of an intersection of the first sound beam and the second sound beam in the acoustic medium. In some embodiments, the location may be relative to a reference location. In some embodiments, the location may be specified in three-dimensional space relative to a reference frame. In some embodiments, properties of the interacting region may include any, any combination of, and/or all of (e.g., at least one of and/or one or more of) a volume of the interacting region, a peak sound pressure of the first sound beam, a peak sound pressure of the second sound beam, a width and depth of a focal region of the first sound beam, and/or a width and depth of a focal region of the second sound beam. In some embodiments, processing the signals to determine properties of the interacting region may include processing the signals to generate an image of one of the first sound beam or second sound beam in the interacting region.

In some embodiments, signals from an ultrasound probe may be received, e.g., by the first and/or second controller and/or control system, to determine location of one or more objects and/or structures in the acoustic medium. In some embodiments, the location may be relative to the second focal point and/or focal region of the second sound beam.

In some embodiments, a first position within a reference frame may correspond to a first source generating the first sound beam and a second position within the reference frame may correspond to a second source generating the second sound beam. In such embodiments, to determine a direction of the first sound beam, a direction of the first sound beam may be adjusted to maximize an amplitude of signals generated from the nonlinear mixing of the first sound beam and the second sound beam. In some embodiments, a position and/or location of a focal point of the second sound beam may be determined based, at least in part, on the first position and direction of the first sound beam.

In some embodiments, an additional (e.g., third) sound beam (e.g., a second probe beam) may be propagated through the acoustic medium, e.g., via an additional (e.g., third) probe beam transducer, e.g., controlled by the first and/or second controller and/or control system. In some embodiments, a third position within the reference frame may correspond to a third source generating a third sound beam, where during adjustment of the direction of the first sound beam the third sound beam may not be propagated. In such embodiments, to determine a direction of the third sound beam, a direction of the third sound beam may be adjusted to maximize an amplitude of signals generated from the nonlinear mixing of the second sound beam and the third sound beam, where during adjustment of the direction of the third sound beam the first sound beam may not be propagated. Further, in some embodiments, a position of the focal point of the second sound beam may be determined based, at least in part, on the first position, the third position, direction of the first sound beam, and direction of the third sound beam.

Embodiments of the present disclosure may be realized in any of various forms. For example, some embodiments may be realized as a computer-implemented method, a computer-readable memory medium (e.g., a non-transitory computer-readable memory medium), and/or a computer system. Other embodiments may be realized using one or more custom-designed hardware devices such as ASICs. Still other embodiments may be realized using one or more programmable hardware elements such as FPGAs.

In some embodiments, a non-transitory computer-readable memory medium may be configured so that it stores program instructions and/or data, where the program instructions, if and/or when executed by a computer system, cause the computer system to perform a method, e.g., any of the method embodiments described herein, or, any combination of the method embodiments described herein, or, any subset of any of the method embodiments described herein, or, any combination of such subsets.

In some embodiments, a computer program, if and/or when executed by a computer system, may cause the computer system to perform a method, e.g., any of the method embodiments described herein, or, any combination of the method embodiments described herein, or, any subset of any of the method embodiments described herein, or, any combination of such subsets.

In some embodiments, a device may be configured to include a processor (or a set of processors) and a memory medium, where the memory medium stores program instructions or a computer program, where the processor is configured to read and execute the program instructions or computer program from the memory medium, where the program instructions are, or computer program is, executable to implement a method, e.g., any of the various method embodiments described herein (or, any combination of the method embodiments described herein, or, any subset of any of the method embodiments described herein, or, any combination of such subsets). The device may be realized in any of various forms.

Although the embodiments above have been described in considerable detail, numerous variations and modifications will become apparent to those skilled in the art once the above disclosure is fully appreciated. It is intended that the following claims be interpreted to embrace all such variations and modifications.

We claim:

1. A method for determining a location of an interacting region of intersecting sound beams within an acoustic medium, comprising:
propagating a first sound beam in a first direction through the acoustic medium from a first location;
adjusting the first direction to maximize an amplitude of signals generated from nonlinear mixing of the first sound beam and a second sound beam propagated through the acoustic medium in a second direction from a second location; and
upon detection of the maximum amplitude of the signals generated from nonlinear mixing of the first sound beam and a second sound beam, determining the location of the interacting region based, at least in part, on the first location and the adjusted first direction.

2. The method of claim 1,
wherein the first location is within a reference frame and corresponds to a first source generating the first sound beam.

3. The method of claim 2,
wherein the first source generating the first sound beam includes a phased-array transducer.

4. The method of claim 1,
wherein the location of the interacting is further based, at least in part, on the second location and the second direction.

5. The method of claim 1, further comprising:
propagating a third sound beam in a third direction from a third location through the acoustic medium; and
adjusting the third direction of the third sound beam to maximize nonlinear mixing of the third sound beam and the second sound beam, wherein during adjustment of the third direction of the third sound beam, the first sound beam is not propagated.

6. The method of claim 5 further comprising:
upon detection of the maximum amplitude of the signals generated from nonlinear mixing of the third sound beam and the second sound beam, determining the location of the interacting region based, at least in part, on the first location, the adjusted first direction, the third location and the adjusted third direction.

7. The method of claim 6,
wherein determining the location of the interacting is further based, at least in part, on the second location and the second direction.

8. The method of claim 1,
wherein the first sound beam has energy concentrated at a first frequency, wherein the second sound beam has energy concentrated at a second frequency, and wherein the signals generated from the nonlinear mixing of the first sound beam and the second sound beam include a sum tone or difference tone.

9. The method of claim 8,
wherein a sum tone corresponds to a sum of the first frequency and the second frequency, wherein the difference tone corresponds to a difference of the first frequency and the second frequency.

10. The method of claim 8,
wherein one of the sum tone or difference tone corresponds to the maximum amplitude of signals generated from nonlinear mixing of the first sound beam and the second sound beam.

11. The method of claim 1, further comprising:
receiving, from at least one sensor, signals resulting from the nonlinear mixing of the first sound beam and the second sound beam, wherein the signals are representative of sound pressure in the acoustic medium; and
processing the signals representative of the sound pressure to determine properties of the interacting region.

12. The method of claim 11,
wherein properties of the interacting region include a location of an intersection of the first sound beam and the second sound beam in the acoustic medium, and wherein the location of the intersection corresponds to a focal point of the second sound beam.

13. The method of claim 1,
wherein adjusting the first direction of the first sound beam within the acoustic medium includes adjusting at least one of:
a frequency of the first sound beam;
an amplitude of the first sound beam;
a depth of a focal point of the first sound beam; or
a focal length of the first sound beam.

14. An apparatus, comprising:
a memory; and
at least one processor in communication with the memory, wherein the at least one processor is configured to:
generate instructions to propagate a first sound beam in a first direction through an acoustic medium from a first location;
generate instructions to adjust the first direction to maximize an amplitude of signals generated from nonlinear mixing of the first sound beam and a second sound beam propagated through the acoustic medium in a second direction from a second location; and
upon detection of the maximum amplitude of the signals generated from nonlinear mixing of the first sound beam and a second sound beam, determine a location of an interacting region based, at least in part, on the first location and the adjusted first direction.

15. The apparatus of claim 14,
wherein the first location is within a reference frame and corresponds to a first source generating the first sound beam.

16. The apparatus of claim 14,
wherein the location of the interacting is further based, at least in part, on the second location and the second direction.

17. The apparatus of claim 14,
wherein the at least on processor is further configured to:
generate instructions to propagate a third sound beam in a third direction from a third location through the acoustic medium; and
generate instructions to adjust the third direction of the third sound beam to maximize nonlinear mixing of the third sound beam and the second sound beam, wherein during adjustment of the third direction of the third sound beam, the first sound beam is not propagated.

18. The apparatus of claim 17,
wherein the at least one processor is further configured to:
upon detection of the maximum amplitude of the signals generated from nonlinear mixing of the third sound beam and the second sound beam, determine the location of the interacting region based, at least in part, on the third location and the adjusted third direction.

19. The apparatus of claim 18,
wherein the location of the interacting is further based, at least in part, on the second location and the second direction.

20. The apparatus of claim 14,
wherein the first sound beam has energy concentrated at a first frequency, wherein the second sound beam has energy concentrated at a second frequency, and wherein the signals generated from the nonlinear mixing of the first sound beam and the second sound beam include a sum tone or difference tone.

21. The apparatus of claim 20,
wherein a sum tone corresponds to a sum of the first frequency and the second frequency, wherein the difference tone corresponds to a difference of the first frequency and the second frequency, and wherein one of the sum tone or difference tone corresponds to the maximum amplitude of signals generated from nonlinear mixing of the first sound beam and the second sound beam.

22. The apparatus of claim 14,
wherein, to generate instructions to adjust the first direction of the first sound beam within the acoustic medium, the at least one processor is configured to generate instructions to adjust at least one of:
a frequency of the first sound beam;
an amplitude of the first sound beam;
a depth of a focal point of the first sound beam; or
a focal length of the first sound beam.

23. An apparatus, comprising:
a memory; and
at least one processor in communication with the memory, wherein the at least one processor is configured to:
receive, from at least one sensor, signals representative of sound pressure in an acoustic medium, wherein the sound pressure results from nonlinear mixing of a first sound beam propagated through the acoustic medium in a first direction and from a first location and a second sound beam propagated through the acoustic medium in a second direction and from a second location; and
process the signals representative of the sound pressure to determine properties of an interacting region of the first sound beam and the second sound beam, wherein the properties of the interacting region include at least a location of an intersection of the first sound beam and the second sound beam.

24. The apparatus of claim 23,
wherein the location corresponds to a maximum amplitude of signals generated from nonlinear mixing of the first sound beam and the second sound beam.

25. The apparatus of claim 23,
wherein the location of the intersection corresponds to a focal point of the second sound beam.

26. The apparatus of claim 23,
wherein the at least one sensor includes at least one of:
a microphone;
a hydrophone; or
an accelerometer.

27. The apparatus of claim 23,
wherein the first sound beam has energy concentrated at a first frequency, wherein the second sound beam has energy concentrated at a second frequency, and wherein the signals generated from the nonlinear mixing of the first sound beam and the second sound beam include a sum tone or difference tone.

28. The apparatus of claim 27,
wherein a sum tone corresponds to a sum of the first frequency and the second frequency, wherein the difference tone corresponds to a difference of the first frequency and the second frequency.

29. The apparatus of claim 27,
wherein one of the sum tone or difference tone corresponds to the maximum amplitude of signals generated from nonlinear mixing of the first sound beam and the second sound beam.

30. The apparatus of claim 23,
wherein properties of the interacting region include at least one of:
a volume of the interacting region;
a peak sound pressure of the first sound beam;
a peak sound pressure of the second sound beam;
a width and depth of a focal region of the first sound beam; or
a width and depth of a focal region of the second sound beam.

31. The apparatus of claim 23,
wherein to process the signals representative of the sound pressure to determine properties of the interacting region, the at least one processor is further configured to process the signals to generate an image of one of the first sound beam or the second sound beam in the interacting region of the acoustic medium.

32. The apparatus of claim 23,
wherein the intersection corresponds to a first location of a first focal point of the first sound beam and a second location of a second focal point of the second sound beam.
